(19) United States
(12) Patent Application Publication
(10) Pub. No.: US 2020/0337798 A1
(43) Pub. Date: Oct. 29, 2020

(54) MEDICAL SAFETY SYSTEM

(71) Applicant: Medi Plus Inc., Tokyo (JP)

(72) Inventors: Naoya Sugano, Tokyo (JP); Minsu Kwon, Tokyo (JP)

(73) Assignee: Medi Plus Inc., Tokyo (JP)

(21) Appl. No.: 16/763,305

(22) PCT Filed: Nov. 1, 2018
(86) PCT No.: PCT/JP2018/040760

§ 371 Date: May 12, 2020

(30) Foreign Application Priority Data
Nov. 20, 2017 (JP) 2017-222866
Jul. 27, 2018 (JP) 2018-141822

(51) Int. Cl. A61B 90/00; G06K 9/46

(52) U.S. Cl. CPC A61B 90/361; G06K 9/4604; G06K 2209/057; A61B 2505/05; A61B 90/39

(57) ABSTRACT

A medical safety system that is more convenient than the related art in view of user-friendliness is provided. The medical safety system includes a server apparatus 120 and a mobile terminal 132. The server apparatus 120 stores a panoramic image generated by imaging an operating room for surgery in a wide-angle manner. The mobile terminal 132 displays a partial image that is part of the panoramic image and identifies, by performing image recognition processing for the panoramic image, an operative field imaged in the panoramic image. The mobile terminal 132 performs, in response to a predetermined impetus, display position adjustment for adjusting the partial image to include the identified operative field.

### TECHNICAL FIELD

The present invention relates to a medical safety system.

### BACKGROUND ART

In recent years, awareness of issues such as medical malpractice and medical error has been raised in society at large, and as a result, demand for information disclosure of medical institutions has been growing. As part of efforts to satisfy such demand from society, a system (hereinafter referred to as a medical safety system in some cases) has been used in part of medical institutions. The system is configured such that a monitoring camera or the like is installed in a facility and images of various events that occur in the facility are imaged and retained as evidential records.

The following patent documents 1 and 2 are examples of related art document disclosing technologies usable for medical safety systems of this kind.

Patent document 1 discloses a technology in which, in accordance with position information selected by a user, part of image is cut out of a full-perimeter monitoring image captured by a monocular camera and an orthoimage is displayed by subjecting the cut-out part, to distortion correction.

Patent document 2 discloses another technology in which moving image data obtained by performing imaging in the street is encoded; when an important part (for example, a pedestrian) imaged in the moving image data is identified, the important part is displayed in an emphasized manner by changing the encoding method for the important part when the moving image data is reproduced.

### CITATION LIST

### Patent Documents

[Patent document 1] Japanese Patent Laid-Open No. 2012-244480
[Patent document 2] Japanese Patent Laid-Open No. 2005-260501

### SUMMARY OF THE INVENTION

### Problem to be Solved by the Invention

Concerning images obtained by, for example, a monitoring camera that aims to continue to capture images of wide areas for long time, users in some cases cannot check details of the images with adequate accuracy due to low resolution, distortion of image caused by wide-angle image capturing, or the like.

Although utilizing the technologies disclosed in the related art documents described above can achieve some improvements in this regard, it cannot yet be said to be sufficient.

The present invention has been made in consideration of the problem described above and provides a medical safety system that is more convenient than the related art in view of user-friendliness.

### Means for Solving the Problem

The present invention provides a medical safety system including a storage unit that stores a panoramic image generated by imaging an operating room for surgery in a wide-angle manner, a display unit capable of displaying a partial image that is a part of the panoramic image, and an identification unit that identifies, by performing image recognition processing for the panoramic image, an operative field imaged in the panoramic image. The display unit performs, in response to a predetermined impetus, display position adjustment for adjusting the partial image to include the operative field identified by the identification unit.

According to the invention, since the display position of a partial image is adjusted to display an operative field identified by performing image recognition processing, it is possible to display the partial image involving the operative field without adjusting the display position by the user while the user views the panoramic image.

### Effect of the Invention

The present invention provides a medical safety system that is more convenient than the related art in view of user-friendliness.

### BRIEF DESCRIPTION OF THE DRAWINGS

FIG. 1 is an illustration depicting a medical safety system according to an embodiment.

FIG. 2 is a perspective view of a hemispherical camera.

FIG. 3 is an illustration depicting a specific example of a panoramic image captured by the hemispherical camera.

FIG. 4 is an illustration depicting a specific example of an image captured by a fixed-point camera.

FIG. 5 is an illustration depicting a specific example of a partial image displayed by a mobile terminal.

FIG. 6 is a schematic illustration for explaining image recognition processing of the mobile terminal.

FIG. 7 is a schematic illustration for explaining image recognition processing of the mobile terminal.

FIG. 8 is a schematic illustration for explaining image recognition processing of the mobile terminal.

FIG. 9 is a schematic illustration for explaining image recognition processing of the mobile terminal.

FIG. 10 provides a specific example of display of a personal computer terminal.

### DESCRIPTION OF EMBODIMENTS

Hereinafter, an embodiment of the present invention is described with reference to the drawings. In all the drawings, almost the same constituent elements are indicated by the same reference characters and description thereof will not be repeated.

### <Constituent Elements Included in Medical Safety System 100>

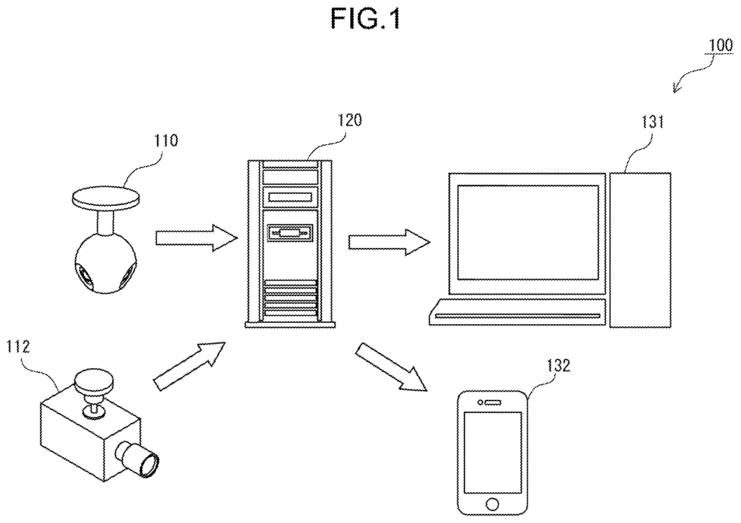

FIG. 1 is an illustration depicting a medical safety system 100 according to the present embodiment.

Arrows illustrated in FIG. 1 each indicate an output source and an input destination between which image data is communicated with respect to constituent elements. Thus, directions in which data other than image data and the like are communicated are not necessarily identical to the transmission and reception directions indicated by the arrows.

The medical safety system 100 includes an imaging unit (for example, a hemispherical camera 111 and a fixed-point camera 112), a server apparatus 120, a viewing terminal apparatus (for example, a personal computer terminal 131 and a mobile terminal 132).

The hemispherical camera 111 is an apparatus that images in a wide-angle manner an operating room for surgery including the field of operation.

Here, the wide-angle image capturing includes obtaining an image of wider area than usual by performing imaging with the use of a monocular wide-angle lens and also includes obtaining an image of wider area than usual by merging a plurality of images captured by performing imaging with the use of a plurality of lenses (standard lenses or wide-angle lenses can be used) that face in directions different from each other.

The hemispherical camera 111 used in the present embodiment is composed of three wide-angle lenses disposed at 120-degree intervals and obtains a single panoramic image by merging, by performing software processing (image processing), three images captured by using the wide-angle lenses. Since such processing is performed, panoramic images obtained by the hemispherical camera 111 performing image capturing are characterized in that the panoramic angle in the horizontal direction reaches 360 degrees.

Thus, by installing the hemispherical camera 111 in an operating room, it is possible to completely capture a full view of the operating room, in which, in addition to the state of an area close to an operative field, motions of medical professionals moving in the operating room and the screen of a medical device displaying vital signs can be imaged at one time. Since it is difficult to falsify images captured in this manner, the authenticity is sufficiently ensured when the images are used as evidential records of circumstances regarding the operation.

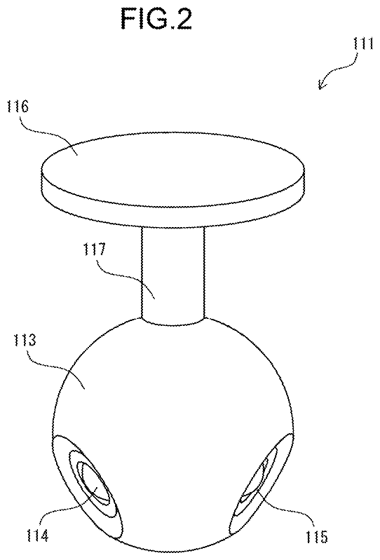

FIG. 2 is a perspective view of the hemispherical camera 111.

The hemispherical camera 111 includes a base 116, a support 117, and a main body 113. The main body 113 has three wide-angle lenses, out of which a lens 114 and a lens 115 are illustrated in FIG. 2.

The main body 113 has a main function of the hemispherical camera 111 (including an imaging function) and is joined to the base 116 by the support 117. It is preferable that the base 116 be positioned above the field of surgical operation; the base 116 may be installed directly at the ceiling of the operating room or installed at a special pole (not shown in the drawings) elongated above the operative field.

As illustrated in FIG. 2, axial directions of the wide-angle lenses (the lenses 114 and 115) provided at the main body 113 are tilted in directions opposite to the base 116, that is, downward directions with respect to the horizontal direction under the precondition that the base 116 positioned above the operative field. Due to such a structure, the hemispherical camera 111 is able to capture a hemispherical image in a downward direction (an image in which the panoramic angle with respect to the horizontal direction reaches 360 degrees and the part in the downward direction is completely imaged). The panoramic image is not necessarily a hemispherical image. The panoramic image may be a full-spherical image (an image in which the panoramic angle reaches 360 degrees with respect to both the horizontal direction and the longitudinal direction) or an image in which the panoramic angle is less than 360 degrees with respect to at least one of the horizontal direction and the longitudinal direction.

The hemispherical camera 111 illustrated in FIG. 2 is an example of a unit that captures panoramic images used in the present invention and an imaging unit is not necessarily included as a constituent element of the present invention. In the case in which an imaging unit is included as a constituent element of the present invention, the imaging unit does not necessarily have the structure described above. For example, the type of lens of the imaging unit is not necessarily of wide-angle lens and the number of lenses of the imaging unit may be increased or decreased.

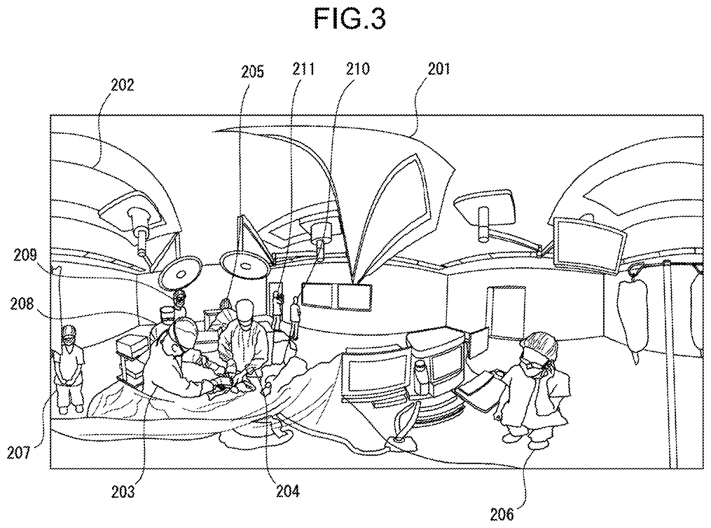

FIG. 3 illustrates a specific example of a panoramic image captured by the hemispherical camera 111.

At the top of the panoramic image, a display device 201 situated close to the ceiling of the operating room and a guide rail 202 that is provided to slide a shadowless lamp, and the like are imaged. As the display device 201 and the guide rail 202 illustrated in FIG. 3, there may be objects distorted as much as the objects cannot be easily recognized.

Additionally, in the panoramic image, a plurality of medical professionals (an operating surgeon 204, an assistant 203, and medical staff members 205 to 211) are imaged. In the following description, these medical professionals are collectively referred to as operators in some cases.

The fixed-point camera 112 is an apparatus that images the field of surgical operation from a position facing to the field of surgical operation. Image capturing by the fixed-point camera 112 only needs to be usual image capturing (does not need to be wide-angle image capturing).

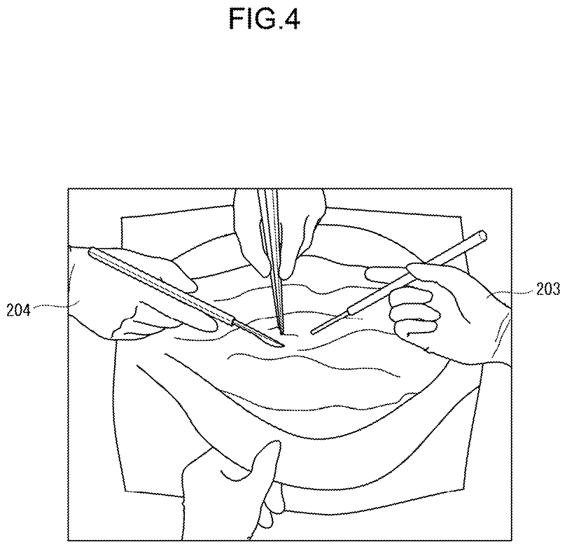

FIG. 4 illustrates a specific example o an image captured by the fixed-point camera 112. As apparent from the comparison between FIGS. 3 and 4, the circumstances (for example, motions of hands of the operating surgeon 204 and the assistant 203) of the operative field can be more clearly viewed in FIG. 4.

In the following description, among images captured by the hemispherical camera 111, an image relating to an operation is in some cases referred to as a “panoramic image”; among images captured by the fixed-point camera 112, an image generated by imaging, from a facing position, an operative field that is part of the imaging range of a panoramic image is in some cases referred to as an “image facing the surgical field”.

As images relating to an operation, a panoramic image s inputted to server apparatus 120 from the hemispherical camera 111 and an image facing the surgical field is inputted to the server apparatus 120 from the fixed-point camera 112. The server apparatus 120 stores the panoramic image and the image facing the surgical field in a predetermined storage area. In this manner, the server apparatus 120 functions as a storage unit of the present invention.

Images stored in the server apparatus 120 may include images obtained from an imaging apparatus or a medical device and the lire not shown in the drawings and these imaging apparatus and medical device may be configured inside or outs/de the medical safety system 100.

The personal computer terminal 131 and the mobile terminal 132 are computer devices in each of which a software application (a viewer) for displaying images stored in the server apparatus 120 is installed.

In the mobile terminal 132, a viewer intended to be used mainly when a medical professional (for example, an anesthetist) waiting outside the operating room checks the ongoing operation in the operating room is installed; the mobile terminal 132 can display images that are stored in the server apparatus 120 and delivered by live streaming.

In the personal computer terminal 131, a viewer intended to be used mainly for analyzing specifics of surgery after the surgery is installed; the personal computer terminal 131 has, in addition to a function of reproducing images stored in the server apparatus 120, a function of editing the images for documents.

The viewers installed in the personal computer terminal 131 and the mobile terminal 132 are not necessarily implemented by software applications especially for the present invention and may be implemented by general software applications or software developed by improving or by modifying the general software applications.

The personal computer terminal 131 and the mobile terminal 132 are both computer devices each including a display device and a pointing device and the type of the display device and the type of the pointing device are not limited to any specific type.

Both the display devices of the personal computer terminal 131 and the mobile terminal 132 can display panoramic images and images facing the surgical field, and additionally, partial images described later, and thus, the display devices can be configured as a display unit of the present invention.

Both the pointing devices of the personal, computer terminal 131 and the mobile terminal 132 can detect a position at which a user's operational input for the display device (for example, various images displayed at the screen is received. The pointing devices of the personal computer terminal 131 and the mobile terminal 132 can be configured as an operational-position detection unit of the present invention.

The function of the personal computer terminal 131 and the function of the mobile terminal 132 described in the present embodiment are not necessarily implemented only the corresponding terminal and part or all of the function of the one of the personal computer terminal 131 and the mobile terminal 132 may be implemented by the other. For example, part or all of the processing of the mobile terminal 132 described later may also be similarly implemented by the personal computer terminal 131.

Furthermore, part or all of the processing of the mobile terminal 132 described later is not necessarily performed by only the mobile terminal 132 and part (for example, image recognition processing) of the processing may be performed by the server apparatus 120.

### <Display of Mobile Terminal 132>

Next, display of the mobile terminal 132 is described.

The mobile terminal 132 is a touch panel capable of obtaining from the server apparatus 120 a panoramic image and an image facing the surgical field that are stored in the server apparatus 120 and displaying individually or together the panoramic image and the image facing the surgical field. The touch panel here denotes a display device in which the screen serves as a pointing device.

The mobile terminal 132 has a function (hereinafter referred to as an identification unit) of identifying, by performing image recognition processing for a panoramic image, a particular area imaged in the panoramic image.

The particular area in the present embodiment is described specifically as an operative field imaged in a panoramic image, but the application of the present invention is not limited to this and another area imaged in a panoramic image may be used as the particular area.

The image recognition processing for identifying an operative field imaged in a panoramic image will be described later.

The mobile terminal 132 has a function (hereinafter referred to as a determination unit) of, when the mobile terminal 132 receives a user's operational input while a panoramic image is displayed, determining whether the position at which the operational input is received is included in the particular area or an operational-input acceptance area that is set close to the particular area.

Here, the operational-input acceptance area is an area that is on the screen of the mobile terminal 132 and that is set in accordance with the processing of the identification unit. The operational-input acceptance area may be involved in the particular area; part of the operational-input acceptance area may overlap the particular area and the remainder may be outside the particular area; or the entire operational-input acceptance area may be outside the particular area and situated close to the particular area.

When the determination result obtained by the determination unit described above is affirmative, the mobile terminal 132 displays an image facing the surgical field. Here, the form of an image facing the surgical field displayed by the mobile terminal 132 in this case is not particularly limited when users can view the image facing the surgical field; the image facing the surgical field may be displayed in the form of a pop-up at an upper layer (a layer) of the panoramic image, in the form in which the image facing the surgical field and the panoramic image are separated in different display areas (windows), or in the form in which the image facing the surgical field is displayed whereas the panoramic image disappears.

As described above, when the position of an operational input received when a panoramic image generated by imaging a relatively wide area is displayed is included in an operative field (the particular area) identified by image recognition processing or an area for determination (an operational-input acceptance area) that is set at a range close to the operative field, the mobile terminal 132 can display an image facing the surgical field in which relatively detailed portions can be easily checked. As a result, the user can obtain necessary information from the image relating to an operation by performing an intuitive operation.

Since the display area of the mobile terminal 132 is smaller than the display area of the personal computer terminal 131, it is difficult to view a panoramic image imaged by the hemispherical camera 111 when the panoramic image is entirely displayed. Thus, the mobile terminal 132 has a function of displaying, in a limiting manner, a partial image that is part of a panoramic image.

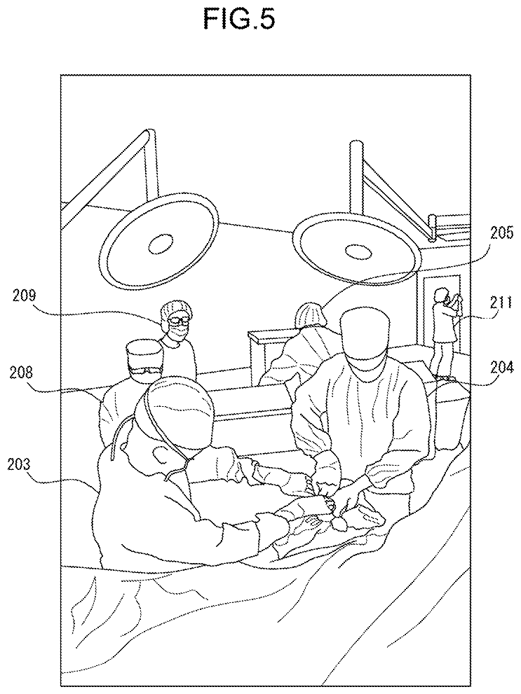

FIG. 5 illustrates a specific example of a partial image displayed by the mobile terminal 132.

As illustrated in FIG. 5, it is preferable that the partial image displayed by the mobile terminal 132 be displayed in a facing manner after the partial image is subjected to distortion correction. This is because this manner enables the user to easily view the partial image.

It is preferable that the display position of a partial image displayed by the mobile terminal 132 can be adjusted by a user operation; it is more preferable that the partial image can cover the full perimeter in at least the horizontal direction when displayed (the display functions as what is called a panorama viewer).

The processing for identifying an operative field imaged in a panoramic image to adjust the display position of a partial image is implemented by the identification unit described above. The mobile terminal 132 has a function of performing, in response to a predetermined impetus, display position adjustment for adjusting the partial image to include an operative field identified by the identification unit.

Here, the predetermined impetus is not particularly limited when the mobile terminal 132 can recognize the impetus (the event) and may be, for example, that the mobile terminal 132 invokes the function of displaying a partial image or that the mobile terminal 132 receives a particular operation. It is preferable, in view of user-friendliness, that the particular operation treated as the predetermined impetus be a simple operation (for example, the one that can be completed by a single operation).

As described above, since the mobile terminal 132 has the function of matching, automatically in response to the predetermined impetus, the display position to an operative field, it is possible to avoid laborious work and time that the user carries out or takes to search for an operative field while viewing a partial image. Because the operative field is one of the parts that should be particularly paid attention to in a panoramic image of an operating room, the function of the mobile terminal 132 for matching the display position to an operative field is very useful in view of user-friendliness.

### <Image Recognition Processing for Identifying Operative Field Imaged in Panoramic Image>

The image recognition processing of the mobile terminal 132 mentioned above is described in detail.

The present inventors decided to use a method of identifying an operative field by performing image recognition processing of detecting a part of the body of an operator for the purpose of making the image recognition processing as a general one. This is because a surgical operation is usually performed by a plurality of operators working on a team and there is thus little possibility that no object targeted to be processed exist in such image recognition processing.

It can be considered that, in the case of specializing in operations performed by using a particular surgical instrument or a particular medical device (including a medical robot), the surgical instrument or the medical device is detected by performing image recognition processing instead of or in addition to a part of the body of an operator.

In the present embodiment, “detecting a part of the body of an operator” is not limited to processing of performing detection while focusing on only an actual part of the body of an operator but may include, for example, detecting eyes of an operator by detecting protective eyewear, detecting the head of an operator by detecting a surgical cap, and detecting the mouth of an operator by detecting a surgical mask.

The method of “image recognition processing for detecting a part of the body of an operator” can be selected as appropriate. Although much trial and error by the present inventors resulted in that a method of extracting a shape (an outline) of a part of the body indicates the most general applicability, high detection accuracy may be achieved in operations performed by using a shadowless lamp by extracting a part of the body while additional attention is paid to colors and luminance regarding an operative field. Moreover, depending on the type of a target part of the body, it can be considered that the part of the body is extracted while additional attention is paid to the motion (the motion pattern) of the part.

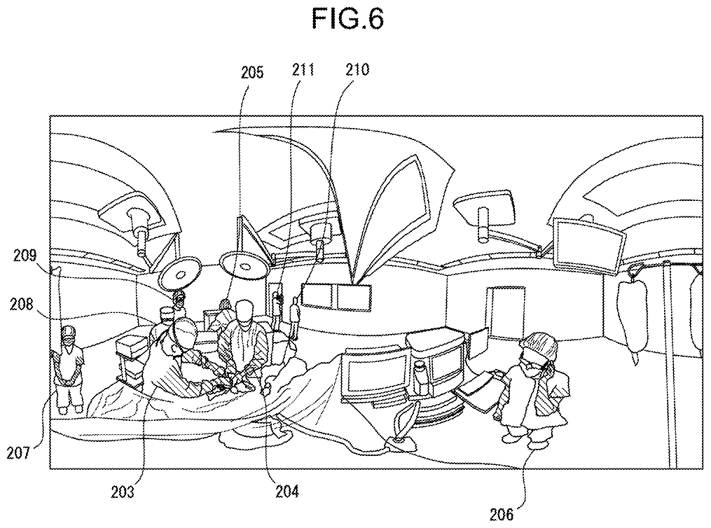

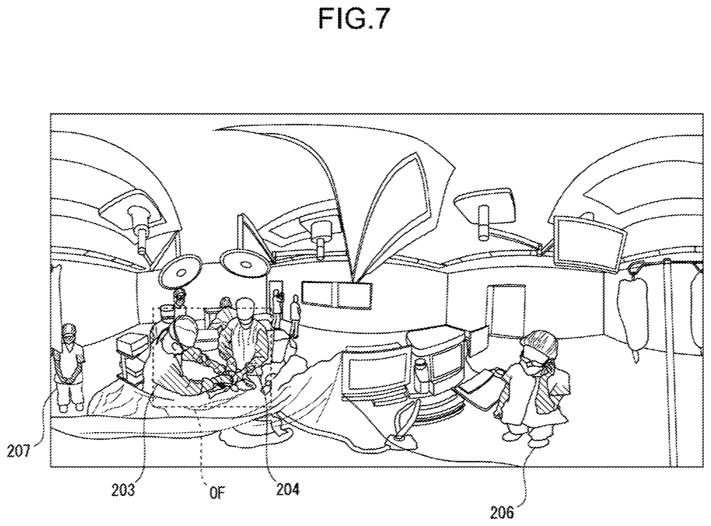

A specific example of the image recognition processing of the mobile terminal 132 is described with reference to FIGS. 6 and 7.

FIGS. 6 and 7 are schematic illustrations for explaining the image recognition processing of the mobile terminal 132, which are different from images actually displayed. In the description, the portions indicated by cross-hatching in these illustrations are parts of the bodies detected by performing image recognition processing of the mobile terminal 132.

In this specific example, a part of the body detected by the mobile terminal 132 is hands or arms of an operator.

For example, it is assumed that, by performing image recognition processing for the panoramic image illustrated in FIG. 3, hands and arms of the operating surgeon 204, the assistant 203, and the medical staff members 206 and 207 are detected (refer to FIG. 6).

Here, hands and arms of the medical staff members 205, 208, and 209 are not sufficiently imaged because the hands and arms of the medical staff members 205, 208, and 209 are hidden behind other objects, and as a result, it is impossible to detect the hands and arms by the image recognition processing. In addition, the medical staff members 210 and 211 are situated apart from the hemispherical camera 111 and not imaged in a sufficient size, and as a result, it is impossible to detect the hands and arms by the image recognition processing.

As described above, when a plurality of parts of the bodies imaged in the panoramic image are detected by the image recognition processing, the mobile terminal 132 identifies as an operative field OF an area close to a position at which the detected hands and arms (the parts of the bodies) densely exist (refer to FIG. 7).

Here, an area close to the operating surgeon 204 and the assistant 203 is the operative field OF.

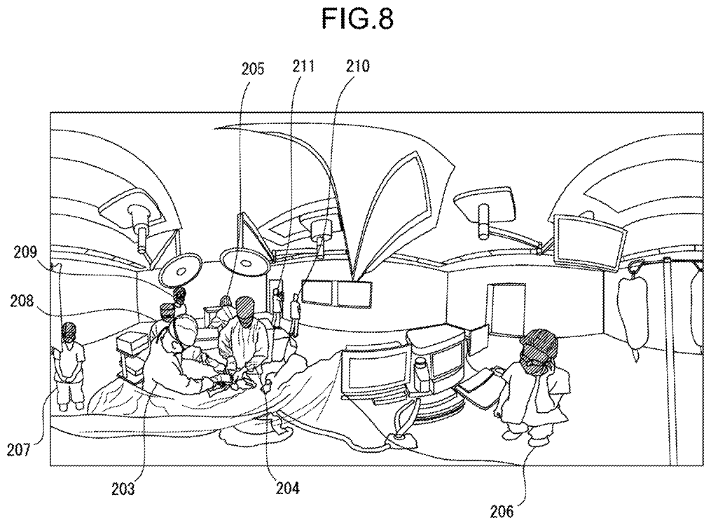

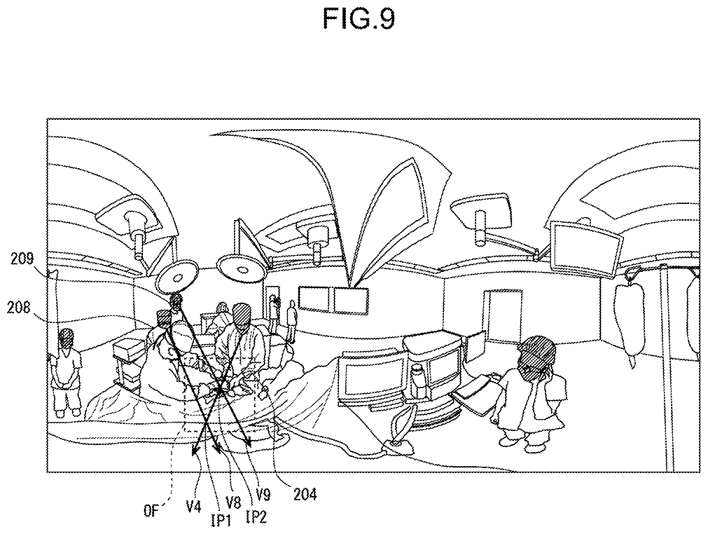

Next, another specific example different from the image recognition processing described above is explained with reference to FIGS. 8 and 9.

Similarly to FIGS. 6 and 7, FIGS. 8 and 9 are schematic illustrations for explaining the image recognition processing of the mobile terminal 132, which are different from images actually displayed. In the description, the portions indicated by cross-hatching in these illustrations are parts of the bodies detected by performing image recognition processing of the mobile terminal 132.

In this specific example, a part of the body detected by the mobile terminal 132 is a face (a head) of an operator and image recognition processing in which both eves are used as a feature is performed to detect the face.

For example, it is assumed that, by performing image recognition processing for the, panoramic image illustrated in FIG. 3, the face of the operating surgeon 204, the faces of the medical staff members 706 to 709 are detected (refer to FIG. 8).

Here, since the face of the assistant 703 faces sideways and the medical staff member 205 faces backward, both eyes are thus not imaged; and thus, it is impossible to detect the faces by the image recognition processing. In addition, the medical staff members 210 and 211 are situated apart from the hemispherical camera 111 and not imaged in a sufficient size, and as a result, it is impossible to detect the faces by the image recognition processing.

The mobile terminal 132 determines the position and the facing direction of the operator in accordance with the detected face and both eyes; when a plurality of operators are detected and the degree of proximity of determined positions of the plurality of operators is equal to or less than a predetermined value, the portion at which the facing directions of the plurality of operators cross each other is identified as an operative field (refer to FIG. 9).

Here, the mobile terminal 132 detects, as operators close to each other, the operating surgeon 204 and the medical staff members 208 and 209 and determines sight-line directions V4, V8, and V9 as the facing directions of the respective operators. The mobile terminal 132 identifies as the operative field OF an area involving the position of an intersection point IP1 of the sight-line directions V4 and V8 and the position of an intersection point IP2 of the sight-line directions V4 and V9. Since the sight-line directions V8 and V9 do not cross each other, the sight-line directions V8 and V9 are not used for identifying the operative field OF.

As apparent from the comparison between FIGS. 7 and 9, when image recognition processing is performed for a particular panoramic image as a target, the identified position of the operative field OF in the particular panoramic image may vary depending on the method of image recognition processing.

Thus, it can be considered to increase the accuracy of identifying an operative field by changing the method of image recognition processing performed by the mobile terminal 132 or combining methods with each other, as appropriate.

### <Display of Personal Computer Terminal 131>

Next, display of the personal computer terminal 131 is described.

Since the display area of the personal computer terminal 131 is larger than the display area of the mobile terminal 132, the display area of the personal computer terminal 131 is capable of displaying an entire panoramic image captured by the hemispherical camera 110. Furthermore, the personal computer terminal 131 can display another image together with the panoramic image.

It should be noted that the above description does not lead to a denial that the personal computer terminal 131 displays a partial image (functions as a panorama viewer for a panoramic image) similarly to the mobile terminal 132 described above.

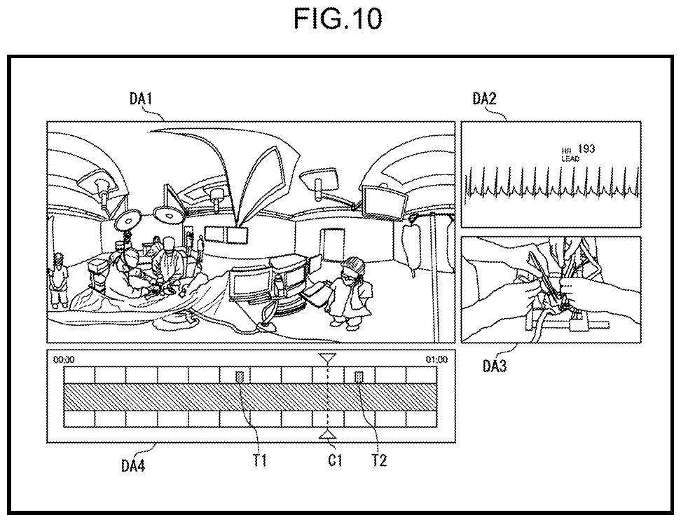

FIG. 10 provides a specific example of display of the personal computer terminal 131.

In a display area DA1, an entire panoramic image of an operating room captured by the hemispherical camera 110 is displayed.

In a display area DA2, an image regarding a heart rate monitor about a patient having had an operation in the operating room is displayed.

In a display area DA3, an image of an operative field of the operation captured by the imaging apparatus not shown in the drawings is displayed.

The personal computer terminal 131 displays these images in a synchronized manner, and as result, it is possible to analyze the operation while the state of the entire operating room, the state of the operative field, and changes in heart rate are compared with each other.

Additional in a display area DA4, a timeline relating to the panoramic image is displayed. Thus, the personal computer terminal 131 functions as a timeline display unit according to the present invention.

A cursor C1 displayed in the display area DA4 indicates where (which time point) the image at present displayed in the display area DA1 is in the timeline.

Tags T1 and T2 displayed in the display area DA4 each indicate, by the display position in the timeline, a particular time at which a beep was sounded, and by the display appearance (for example, colors), a particular hardware device that sounded the beep. The beep here denotes a sound (for example, an alarm sound) sounded by a hardware device.

The hemispherical camera 110 can record sound data by using a microphone (not shown in the drawings) while performing capturing a panoramic image.

The server apparatus 120 stores the recorded sound data in association with image data of the corresponding panoramic image. Furthermore, the server apparatus 120 has a function of detecting a beep contained in the sound data and specifying the time of the beep by performing sound recognition processing for the sound data and also has a function of identifying a hardware device that sounded the detected beep. Thus, the server apparatus 120 functions as a beep identification unit according to the present invention.

The personal computer terminal 131 enables the user to recognize the time of a beep detected by the server apparatus 120 and a hardware device that sounded the beep by displaying the tags T1 and T2.

Since the sounded beep varies depending on the hardware device or the manufacturer of the hardware device, it is possible to recognize what kind of incident has occurred and what time the particular incident has occurred by recording and analyzing the beep.

However, a medical safety system having the above-described functions of recording a beep together with image data for analysis, recognizing the beep by performing sound recognition processing, and displaying the time of the beep has not been introduced into medical settings. Because the medical safety system 100 according to the present embodiment has these functions, it is possible to use the medical safety system 100 for, for example, discovering the cause when a medical error has happened.

### MODIFIED EXAMPLES OF PRESENT INVENTION

While the present invention has been described in accordance with the embodiment explained with reference to the drawings, the present invention is not limited to the embodiment described above and encompasses various modes such as modifications and improvements when the object of the present invention can be achieved.

It should be noted that, in modified examples described below, when a function is described as the function of the personal computer terminal 131 or the mobile terminal 132, the function is not necessarily implemented by only the corresponding terminal and part or all of the function described as the function of the one of the personal computer terminal 131 and the mobile terminal 132 may be implemented by the other.

While the embodiment described above is explained on the basis of the constituent elements illustrated in FIG. 1, constituent elements of the present invention only have to be formed to implement functions of the constituent elements. Thus, the constituent elements of the present invention do not need to exist individually, and it is allowed, for example, to form a plurality of constituent elements as a single member, to form a single constituent element including a plurality of members, to Include a constituent element in another constituent element, and to enable part of a constituent element and part of another constituent element to exist in a duplicated manner.

For example, the medical safety system according to the present invention does not necessarily include an imaging apparatus corresponding to the hemispherical camera 111; the present invention may be implemented by using a panoramic image obtained by an imaging apparatus outside the system.

The configuration of the hemispherical camera 111 and the imaging method of the hemispherical camera 111 described above are a mere specific example and the implementation of the present invention is not limited to this.

For example, the present invention may be implemented by using an imaging apparatus employing a monocular wide-angle lens and panoramic images captured by this imaging apparatus.

While in the embodiment described above hands, arms, and a head are used as specific examples of a part of the body of an operator detected by the mobile terminal 132, another part may be detected instead of or in addition to these.

While in the embodiment described above the case in which the mobile terminal 132 detects the face of an operator by using both eyes as a feature is explained, the position and the facing direction of an operator may be determined by detecting the face by performing image recognition processing using another part (a nose, a mouth, ears, or the like) as a feature.

While in the embodiment described above the example in which one candidate for the operative field identified by the mobile terminal 132 performing image recognition processing is explained, when a plurality of identified candidates for the operative field exist, the user may be offered options and an area close to an operative field selected by the user may be displayed. Alternatively, in such a case, the mobile terminal 132 may display a particular candidate while changing the candidate among a plurality of candidates whenever a particular operation is received.

While in the embodiment described above the particular area identified by the image recognition processing performed by the mobile terminal 132 is only an operative field, a function of identifying another particular area may be included. For example, in the case in which the panoramic image is an image generated by imaging an operating room including a medical device used for the operation and the image captured by the fixed-point camera 112 is an image generated by performing imaging from an angle facing to the medical device, the mobile terminal 132 (the identification unit) may be able to identify as the particular area the position of the medical device imaged in the panoramic image.

In this case, the mobile terminal 132 (the identification unit) may detect, by performing image recognition processing, a plurality of markers imaged in the panoramic image and identify the position of the medical device in accordance with the positions of the plurality of detected markers (for example, markers are attached to four corners of the screen of the medical device before the surgery is performed and a rectangular area surrounded by the plurality of markers is identified as the position of the medical device).

Alternatively, the mobile terminal 132 can recognize one or a plurality of medical devices with respect to the shape and colors by performing pattern recognition and may identify, as a particular medical device, one of the objects imaged in a panoramic image, the one indicating matching in the pattern recognition.

While the determination unit according to the embodiment described above is explained as the one that determines whether the position at which a user's operational input is received while a panoramic image is displayed is included in the operational-input acceptance area involved in the panoramic image, the determination unit may determine whether the position at which a user's operational input is received is included in the operational-input acceptance area of a partial image also in the case of displaying the partial image described above.

In this modified example, in the case in which the image captured by the fixed-point camera 112 is an image facing the surgical field and the particular area identified by the identification unit is an operative field, when the determination result obtained by the determination unit is affirmative, the display unit may display an image facing the surgical field.

Furthermore, in this modified example, in the case in which the image captured by the fixed-point camera 112 is an image captured from an angle facing to a medical device and the particular area identified by the identification unit is the position of the medical device, when the determination result obtained by the determination unit is affirmative, the display unit may display a facing image of the medical device.

Moreover, in this modified example, in the case in which the image captured by the fixed-point camera 112 is an image facing the surgical field or a particular image other than an image facing the medical device and the particular area identified by the identification unit is an imaging area of an object imaged in the particular image, when the determination result obtained by the determination unit is affirmative, the display unit may display the particular image.

While in the description of the embodiment described above the personal computer terminal 131 displays, in the display area DA4, a timeline regarding a panoramic image displayed in the display area DA1, the personal computer terminal 131 may display, additionally in the display area DA4, timelines regarding other images displayed in the display areas DA2 and DA3. This means that the personal computer terminal 131 may display timelines individually regarding a plurality of images displayed in a synchronized manner.

The present embodiment encompasses the following technical ideas.
(1-1) A medical safety system including a storage unit that stores a panoramic image generated by imaging an operating room for surgery in a wide-angle manner, a display unit capable of displaying a partial image that is a part of the panoramic image, and an identification unit that identifies, by performing image recognition processing for the panoramic image, an operative field imaged in the panoramic image, wherein the display unit performs, in response to a predetermined impetus, display position adjustment for adjusting the partial image to include the operative field identified by the identification unit.
(1-2) The medical safety system according to (1-1), further including an operational-position detection unit, capable of detecting a position at which an operational input by a user for the display unit is received and a determination unit that, when the operational-position detection unit receives the operational input by the user while the display unit displays the panoramic image or the partial image, determines whether the position at which the operational input is received is included in the operative field identified by the identification unit or an operational-input acceptance area that is set close to the operative field, wherein the display unit is capable of displaying, in addition to the partial image, the panoramic image and an image facing the surgical field generated by performing imaging from an angle facing to the operative field, and the display unit displays the image facing the surgical field when a determination result obtained by the determination unit is affirmative.
(1-3) The medical safety system according to (1-1) or (1-2), wherein the identification unit identifies the operative field by performing image recognition Processing of detecting a part cf the body of an operator.
(1-4) The medical safety system according to (1-3), wherein the part of the body detected by the identification unit is a hand or arm of the operator and, and when the identification unit detects a plurality of parts of the bodies, each being the part of the body, that are imaged in the panoramic image, the identification unit identifies as the operative field a position at which the plurality of detected parts of the bodies densely exist.
(1-5) The medical safety system according to (1-3), wherein the identification unit determines a position of the operator and a facing direction of the operator in accordance with the detected part of the body, and when a plurality of operators are detected and a degree of proximity of determined positions of the plurality of operators is equal to or less than a predetermined value, the identification unit identifies as the operative field an area close to a position at which facing directions of the plurality of operators cross each other.
(1-6) The medical safety system according to any one of (1-1) to (1-5), wherein the panoramic image stored in the storage unit is an image that is obtained by merging a plurality of images generated by performing imaging in directions different from each other and in which a panoramic angle in a horizontal direction reaches 360 degrees.
(1-7) The medical safety system according to any one of (1-1) to (1-6), wherein the storage unit stores, in association with image data of the panoramic image, sound data recorded while the panoramic image is imaged, and a beep identification unit is included, the beep identification unit being configured to, by performing sound recognition processing for the sound data, detect a beep contained in the sound data and identify a hardware device that sounded the detected beep.
(1-8) The medical safety system according to (1-7), including a timeline display unit that displays a timeline regarding the panoramic image, wherein the timeline display unit displays in the timeline a time at which the beep detected by the beep identification unit was sounded.
(2-1) A medical safety system including an input unit that inputs a first image regarding an operation and a second image generated by imaging part of the imaging range of the first image, a display unit capable of displaying the first image and the second image that are inputted by the input unit, an operational-position detection unit capable of detecting a position at which an operational input by a user for the display unit is received, an identification unit that identifies a particular area imaged in the first image by performing image recognition processing for the first image, and a determination unit that, when the operational-position detection unit receives the operational input while the display unit displays the first image, determines whether the position at which the operational input is received is included in the particular area or an operational-input acceptance area that is set close to the particular area, wherein the display unit displays the second image when the determination result obtained by the determination unit is affirmative.
(2-2) The medical safety system according to (2-1), wherein the first image is an image generated by imaging an operating room including an operative field, the second image is an image generated by imaging the operative field from a facing angle, and the identification unit identifies as the particular area the operative field imaged in the first image.
(2-3) The medical safety system according to (2-1), wherein the first image is an image generated by imaging an operating room including a medical device used for an operation, the second image is an image generated by performing imaging from an angle facing to the medical device, and the identification unit identifies as the particular area the position of the medical device imaged in the first image.
(2-4) The medical safety system according to (2-3), wherein the identification unit detects, by performing image recognition processing, a plurality of markers imaged in the first image and identifies the particular area in accordance with the positions of the plurality of detected markers.
(2-5) The medical safety system according to any one of (2-1) to (2-4), wherein the first image is an image that is obtained by merging a plurality of images generated by performing imaging in directions different from each other and in which a panoramic angle in a horizontal direction reaches 360 degrees.

This application claims priority based on Japanese Patent Application No. 2017-222866, filed on Nov. 20, 2017, and Japanese Patent Application No. 2018-141822, filed on Jul. 27, 2018 and the disclosure thereof is incorporated herein in its entirety.

### REFERENCE SIGNS LIST

100 medical safety system

111 hemispherical camera

112 fixed-point camera

120 server apparatus

131 personal computer terminal

132 mobile terminal

201 display device

202 guide rail

203 assistant

204 operating surgeon

205 to 211 medical staff member

1. A medical safety system comprising:
a storage unit that stores a panoramic image generated by imaging an operating room for surgery in a wide-angle manner;
a display unit capable of displaying a partial image that is a part of the panoramic image; and
an identification unit that identifies, by performing image recognition processing for the panoramic image, an operative field imaged in the panoramic image, wherein
the display unit performs, in response to a predetermined impetus, display position adjustment for adjusting the partial image to include the operative field identified by the identification unit.

2. The medical safety system according to claim 1, further comprising:
an operational-position detection unit capable of detecting a position at which an operational input by a user for the display unit is received; and
a determination unit that, when the operational-position detection unit receives the operational input by the user while the display unit displays the panoramic image or the partial image, determines Whether the position at which the operational input is received is included in the operative field identified by the identification unit or an operational-input acceptance area that is set close to the operative field, wherein
the display unit is capable of displaying, in addition to the partial image, the panoramic image and an image facing the surgical field generated by performing imaging from an angle facing to the operative field, and
the display unit displays the image facing the surgical field when a determination result obtained by the determination unit is affirmative.

3. The medical safety system according to claim 1, wherein the identification unit identifies the operative field by performing image recognition processing of detecting a part of a body of an operator.

4. The medical safety system according to claim 3, wherein
the part of the body detected by the identification unit is a hand or arm of the operator, and
when the identification unit detects a plurality of parts of bodies, each being the part of the body, that are imaged in the panoramic image, the identification unit identifies as the operative field a position at which the plurality of detected parts of the bodies densely exist.

5. The medical safety system according to claim 3, wherein
the identification unit determines a position of the operator and a facing direction of the operator in accordance with the detected part of the body, and
when a plurality of operators are detected and a degree of proximity of determined positions of the plurality of operators is equal to or less than a predetermined value, the identification unit identifies as the operative field an area close to a position at which facing directions of the plurality of operators cross each other.

6. The medical safety system according to claim 1, wherein
the panoramic image stored in the storage unit is an image that is obtained by merging a plurality of images generated by performing imaging in directions different from each other and in which a panoramic angle in a horizontal direction reaches 360 degrees.

7. The medical safety system according to claim 1, wherein
the storage unit stores, in association with image data of the panoramic image, sound data recorded While the panoramic image is imaged, and
a beep identification unit is included, the beep identification unit being configured to, by performing sound recognition processing for the sound data, detect a beep container in the sound data and identify a hardware device that sounded the detected beep.

8. The medical safety system according to claim 7, comprising:
a timeline display unit that displays a timeline regarding the panoramic image, wherein
the timeline display unit displays in the timeline a time at which the beep detected by the beep identification unit was sounded.
